(19) United States
(12) Patent Application Publication
(10) Pub. No.: US 2006/0015060 A1
(43) Pub. Date: Jan. 19, 2006

(54) Device for treatment of wounds in body tissue via exposure to gases

(76) Inventor: Tony D. Oglesby, Swainsboro, GA (US)

Correspondence Address: FISH & RICHARDSON P.C., P.O. BOX 1022, MINNEAPOLIS, MN, 55440-1022, US

(21) Appl. No.: 10/894,278

(22) Filed: Jul. 19, 2004

(51) Int. Cl. A61M 37/00

(52) U.S. Cl. 604/23

(57) ABSTRACT

A wound treatment device comprising an enclosed space formed from the joining of two layers of material to form a main unit is used to provide and maintain oxygen-rich or oxygen-depleted microenvironments over a wound. At least one inlet connects at one end to a source for gas and, at an opposite end, to the enclosed space to provide the main unit with gas to be delivered to a wound for treatment thereof. The plurality of delivery openings provides uniform delivery of one or more gases to the microenvironment associated with the wound. Gas is exhausted from the microenvironment associated with the wound to the external environment via the plurality of exhaust outlets such that the exhausted gases do not contaminate or otherwise interfere with the delivery of one or more gases to the microenvironment associated with the wound.

### TECHNICAL FIELD

This invention relates to wound treatment devices, and more particularly to an improved device for treatment of wounds in body tissue via exposure to gases.

### BACKGROUND

The invention relates to a device for treatment of wounds in body tissue via exposure of the area of the wound to one or more gases.

Treating a wound with gas (e.g., oxygen) in order to promote and expedite healing of the wound and reduce associated discomfort, such as itching, is generally known. For example, topical hyperbaric oxygen therapy (HBOT) is a technique of delivering 100% oxygen directly to an open, moist wound at a pressure slightly higher than atmospheric pressure. It is theorized that the high concentrations of oxygen diffuse directly into the wound to increase the local cellular oxygen tension, which in turn, promotes healing. Typically, such treatment is accomplished via direct application of gas to the wound area using a hose or similar delivery device aimed at the wound area. This approach requires manual attention by staff or caregivers to position and hold the hose during treatment. Additionally, application of the gas is inconsistent and can vary with each manual application, thus potentially reducing the beneficial effects of the treatment. The relative expense and lack of consistency associated with this form of treatment is therefore disfavored.

Another method by which to introduce gas for the purpose of wound treatment is via systemic hyperbaric oxygen therapy (HBOT). Systemic HBOT is a technique of delivering higher pressures of oxygen internally to tissues. In systemic HBOT, the patient is entirely enclosed in a pressure chamber (e.g., hyperbaric chamber) and breathes oxygen at a pressure greater than 1 atmosphere (the pressure of oxygen at sea level). This technique relies on systemic circulation to deliver highly oxygenated blood to target tissues. Systemic HBOT is used to treat wounds as well as systemic illnesses such as air or gas embolism, carbon monoxide poisoning, and clostridial gas gangrene. Such hyperbaric chambers are substantially sealed enclosures where the inside environment of the chamber provides concentrations of various gases (e.g., oxygen). Although providing some level of consistency of treatment, the use of hyperbaric chambers and the like is relatively expensive, lacks portability, and is overly complex for the type of treatment typically needed for expedited wound healing.

Additional disadvantages associated with known oxygen delivery devices include: inability to provide more than one gas source to the device without pre-mixing the gases (i.e., such devices only have one inlet for gas); non-disposability, use of inferior materials (e.g., non-biodegradable, non-hypoallergenic), no protection against multiple uses, and difficulty of use (e.g., placement/fixing of device).

On balance, the controlled and consistent, topical delivery of gas(es) to a wound area is preferred for reasons associated with relative costs and complexity. The present invention provides an improved wound treatment device for such use.

### SUMMARY

An improved wound treatment device that provides and maintains oxygen-rich or oxygen-depleted microenvironments over a wound is disclosed. The present invention device is comprised of a main unit having an enclosed space therein, one or more inlets, a plurality of delivery openings, and a plurality of exhaust outlets. The one or more inlets of the main unit are connected to one or more sources of gas, and are designed to provide the enclosed space with one or more pressurized gases. As the inlets provide pressurized gas to the main unit, the enclosed space is temporarily inflated. The plurality of delivery openings allows the gas within the enclosed space to be provided directly and evenly to the wound area of the user. The plurality of exhaust outlets allows gases to escape the immediate environment of the wound area directly to the outside environment, without passing through the enclosed space.

In a preferred embodiment, the device of the present invention is a single-use device and is disposable and biodegradable. This feature greatly reduces the chances of multiple uses of a treatment device on wounds by including a feature that compromises the integrity of the material of the treatment device upon removal after treatment. More than a single use of a wound treatment device, even on the same wound, much less on different wounds, or, worse, different patients, can result in the spread of microorganisms (e.g., bacteria) that may actually harm the user via additional or prolonged infection, for example.

The placement and distribution of the plurality of outlets and the plurality of delivery openings over the surface of the main unit is important to the proper function of a wound treatment device. One of the improvements of the present invention device over known devices lies in the number, placement and distribution of such outlets and delivery openings. Specifically, the delivery openings of the present invention device are greater in number and are distributed over a larger area of the main unit. This feature provides several advantages to the device. First, the more widely distributed delivery openings provide a more even environment of gas delivered to the wound area, thereby increasing the consistency of the treatment. With fewer delivery openings and/or delivery openings spaced too closely together, “hot spots” of high gas concentrations can form within the field being treated. Additionally, the larger number and wider distribution of delivery openings restricts the amount of inflation of the enclosed space of the main unit. Less inflation equates to less deformation of the general shape and size of the main unit, thereby markedly decreasing the chances of disrupting the adherence of the main unit to the area surrounding the wound during treatment.

In a preferred embodiment of the present invention device, the main unit is constructed from a hypoallergenic, biodegradable, disposable, flexible material exhibiting relatively low gas permeability. Additionally, the material includes select regions capable of removably adhering to the area surrounding the wound to be treated or is capable of be mated with material capable of providing such removable adherence. Such adherence assists in the formation of a barrier around the wound and temporarily fixes the device in place, eliminating the need for, and inconsistency of treatment inherent with, manual placement and manipulation of the device during treatment. In a preferred embodiment, the integrity of the material is compromised upon its removal to prevent multiple uses of the device. Compromising the material can be accomplished in one of any number of ways, such as select destruction of portions of the material and the like.

In a preferred embodiment, the one or more inlets of the device are fitted with a connector that allows for surgical tubing of any appropriate length to be used. This feature of the device allows the device to be used at varying distances remote from the one or more gas sources (e.g., gas tank). Prior art devices are often fitted with predetermined lengths of tubing that require the user and/or the source of gas to be moved to accommodate use of the device.

In another embodiment, a porous, non-adhering dressing material, such as gauze, will be used in connection with the main unit to provide additional protection to the wound.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a bottom view of one embodiment of the wound treatment device of the present invention;

FIG. 2 is a top view of the embodiment of FIG. 1 of the wound treatment device of the present invention;

FIG. 3 is a top view of another embodiment of the wound treatment device of the present invention; and

FIG. 4 is a cross section of an embodiment of an embodiment of the wound treatment device of the present invention positioned over a wound to be treated.

Like reference symbols in the various drawings indicate like elements.

### DETAILED DESCRIPTION

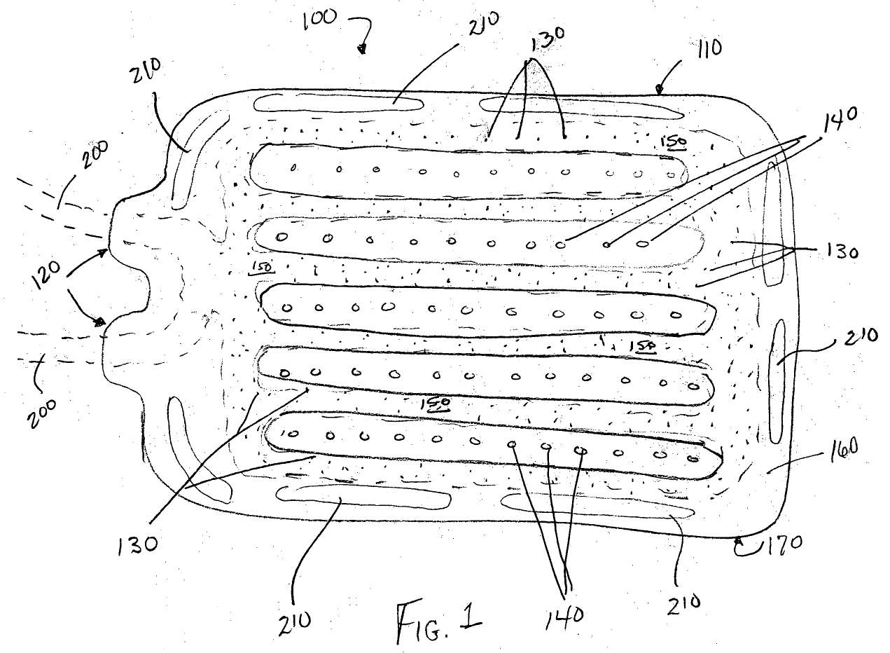

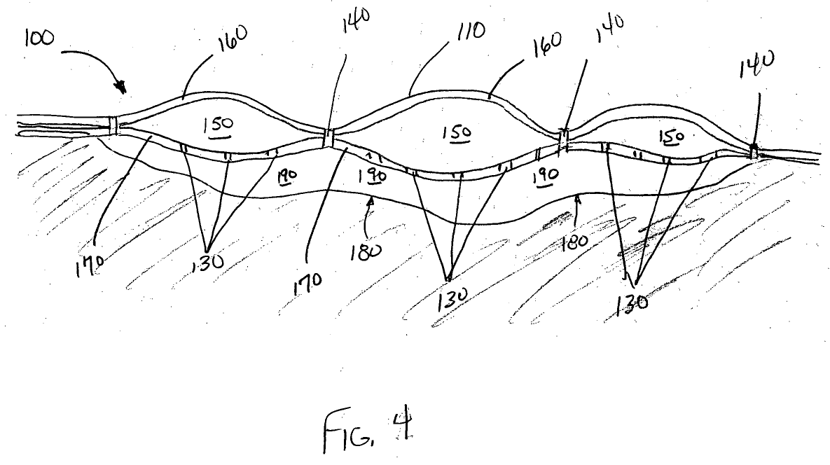

Referring now to FIG. 1, an improved wound treatment device 100 that provides and maintains a microenvironment over a wound (FIG. 4) is shown. The wound treatment device 100 comprises a main unit 110, one or more inlets 120, a plurality of delivery openings 130, and a plurality of outlets 140. As more easily seen in FIG. 4, the main unit 110 further includes an enclosed space 150 defined by an upper layer 160 and a lower layer 170 of the main unit 110. When in use, the wound treatment device 100 is placed over a wound 180 to form a microenvironment 190 above the wound 180, using zones 210 (FIG. 1) for removably adhering the main unit 110 to tissue surrounding the wound 180.

Referring to FIG. 4, the upper layer 160 and the lower layer 170 of the main unit 110 are fused together in one or more areas to form “pillows” within the or enclosed space 150. As will be described in greater detail below, the enclosed space 150 facilitates the delivery of the one or more gases for treatment of the wound 180, and assists in exhausting gases from the microenvironment 190 associated with the wound 180. Although the fusing of the upper layer 160 and the lower layer 170 is described herein to provide pillows, the mating of the upper layer 160 with the lower layer 170 of material in any suitable manner may be used with the present invention, if desired.

Referring again to FIG. 1, the one or more inlets 120 are sized and shaped to accept and secure at one end a hose 200, such as surgical tubing and the like, that leads to one or more sources of gas (not shown). At an opposite end, the one or more inlets 120 are in communication with the enclosed space 150 of the main unit 110. The use of more than one inlet 120 allows for a mixture of gases to be introduced into the main unit 110 for delivery to the wound treatment area. This feature allows for wounds to be treated with a mixture of gases without the need for pre-mixing of the gases. The one or more gases to be delivered to the microenvironment 190 associated with the wound 180 may be any suitable gas, such as oxygen, air or nitrogen, and the like, and/or mixtures thereof. In a preferred embodiment, at least one of the gases is oxygen, preferably pure oxygen, as used in various hyperbaric oxygen treatment methods known in the art.

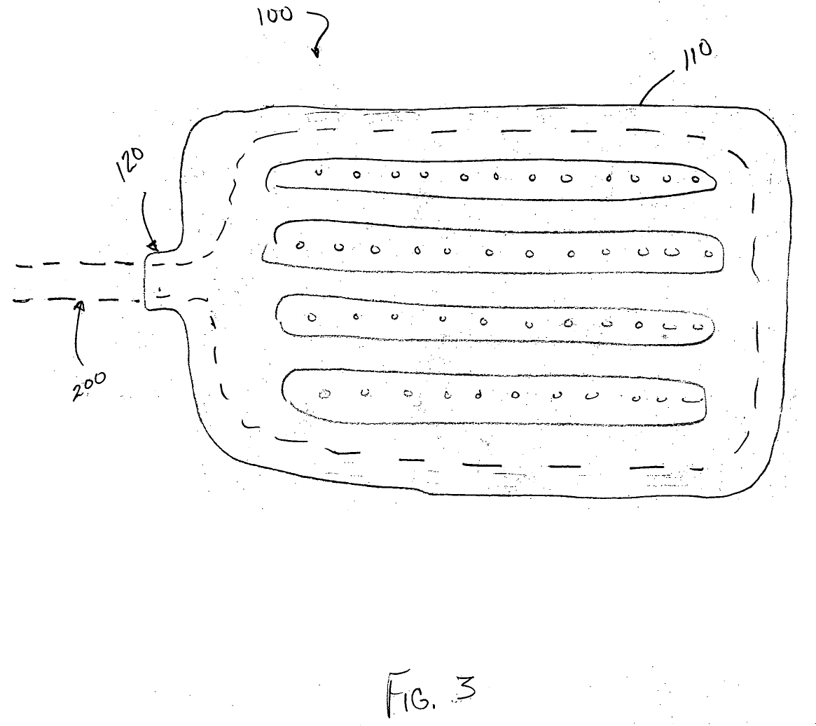

Although two inlets 120 generally located on the same side of the main unit 110 of the wound treatment device 100 are shown and described herein, it is noted that any number and/or sizes and/or shapes of inlets can be used, as desired, with the improved wound treatment device 100 of the present invention. For example, FIG. 3 illustrates an embodiment of the main unit 110 of the improved wound treatment device 100 including a single inlet 120 and a single hose 200 for delivery of gas to the microenvironment 190 associated with the wound 180. Additionally, in a preferred embodiment, the hose 200 is of sufficient length to allow the main unit 110 to be used as remote from the source of gas as is required. The hose 200 can either be supplied in predetermined lengths with the main unit 110, or can be common surgical tubing or the like.

Now referring to FIGS. 1 and 4, the improved wound treatment device 100 includes a plurality of delivery openings 130. The delivery openings 130 are located only within the lower layer 170 of the main unit 110, and are sized and shaped to allow gas to pass from the enclosed space 150 via the delivery openings 130 into the microenvironment 190 surrounding the wound 180. Optimally, the delivery openings 130 are dispersed uniformly substantially throughout the entire surface of the lower layer 170 of the main unit 110 so that gas is delivered in a relatively uniform manner to the microenvironment 190 above the wound 180. In a preferred embodiment, the plurality of delivery openings 130 is arranged in rows along the lower layer 170 of the main unit 110. Although a plurality of delivery openings 130 positioned in rows within the lower layer 170 of the main unit 11 0, spaced to provide a relatively uniform delivery of gas from the enclosed space 150 to the microenvironment 190 associated with the wound 180, is described herein, any number and relative placement of delivery openings 130 are contemplated by the present invention and can be utilized, if desired.

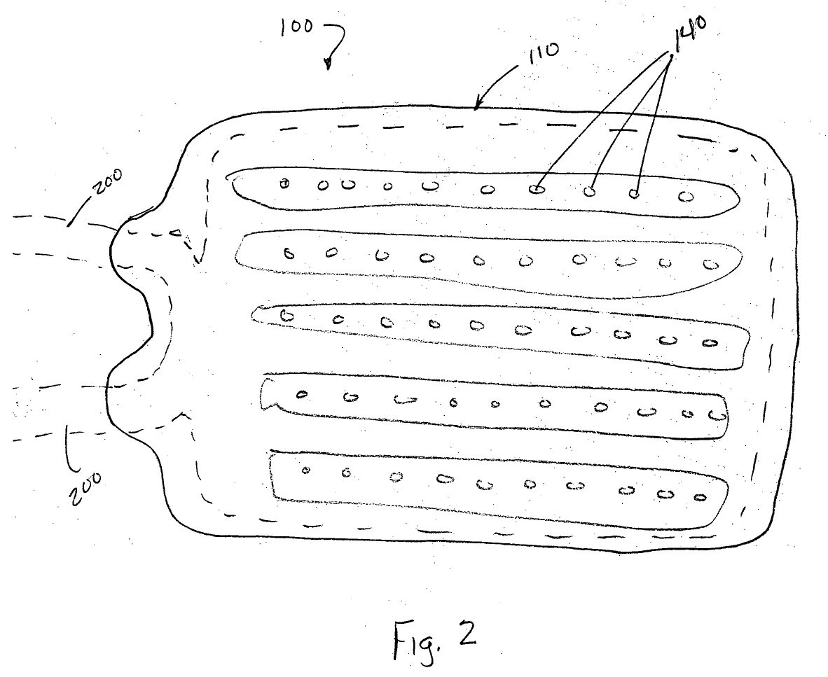

Now referring to FIGS. 2 and 4, also associated with the main unit 110 of the wound treatment device 100 is a plurality of exhaust outlets 140. As shown in FIG. 4, the plurality of exhaust outlets 140 lies at the intersections of the lower layer 170 and the upper layer 160 of the main unit 110, forming the pillows, such that they form a conduit between the microenvironment 190 associated with the wound 180 and the outside environment external to the wound treatment device 100. The exhaust outlets 140 allow for unidirectional fluid (gas) exchange from the microenvironment 190 associated with the wound 180 to the external environment without passing through, and thereby potentially contaminating, or otherwise interfering with, the enclosed space 150. This feature of the wound treatment device 100 of the present invention allows gases to be exhausted from the microenvironment 190 associated with the wound 180 to the external environment without impeding, contaminating, diluting or otherwise interfering with the delivery of the one or more gases via the plurality of delivery openings 130 to the wound 180.

As the one or more inlets 120 provide pressurized gas(s) to the main unit 110, the pillows associated with the enclosed space 150 slightly inflate, providing the microenvironment 190 associated with the wound 180 with the one or more gases via the plurality of delivery openings 130. Having a relatively large number of delivery openings 130 spaced uniformly substantially throughout the entire surface of the lower layer 160 of the main unit, prevents the enclosed space 150 from inflating to a point where the main unit 110 is substantially raised off of its preferred resting position above the wound 180. If there exist too few number of delivery openings 130 and/or the spacing or placement of the delivery openings 130 are such that it causes over-inflation, the main unit 110 can rise away from the wound 180 (FIG. 4) such that the microenvironment 190 associated with the wound 180 is comprised, thereby potentially adversely affecting treatment of the wound 180.

The positive pressure of the one or more gases being provided to the microenvironment 190 associated with the wound 180 prevents backflow of fluid (gas) from the microenvironment 190 to the enclosed space 150, and also facilitates the exhaust of gas(es) via the plurality of outlets 140.

In a preferred embodiment, the upper layer 160 and the lower layer 170 of the main unit 110 of the wound treatment device 100 are constructed from a lightweight, flexible material, such as vapor permeable paper, which is permeable to both water vapour and oxygen, impermeable to micro-organisms, and is disposable, biodegradable and capable of being easily and removably secured to human skin, tissue and the like. Representative materials include OpSite™ wound dressings by Smith Nephew, Inc., and the like.

In another embodiment, a porous, non-adhering dressing material, such as gauze, is fused to the lower layer 170 to provide added protection to the wound bed and periwound area. Representative materials to be used with the lower layer 170 include Aquaphor Gauze™ by Smith & Nephew, Inc., and the like.

The plurality of delivery openings 130 and the plurality of outlets 140 may be formed via selective piercing of the material forming the upper layer 160 and lower layer 170 of the main unit, or in any other manner suitable for the material.

Use of the improved wound treatment device 100 of the present invention obviates the need for health care personnel or any other to position and hold the wound treatment device 100 in place for treatment. This feature is particularly advantageous where wounds require repeated, prolonged treatment regimes.

Preferably, the main unit 110 of the wound treatment device 100 is sterilized and pre-packaged for a single use. Single use is preferred to avoid potential for contamination of wounds to be treated. Such contamination can occur even between wounds of the same person and should be avoided. Use of the preferred embodiment including multiple-use prevention adhesion zones is recommended to prevent such problems. In one embodiment, the zones 210 include a peel and stick adhesive strip that facilitates removable affixing of the wound treatment devices. In a preferred embodiment, the zones 210 of the main unit 110 include means to compromise the integrity of the lower layer 170 of the main unit 110 upon removal of same to prevent multiple uses of the main unit 110. Examples of means to compromise include the use of adhesives that allow for only a single use, such that removal of the zones 210 from the tissue of the user prevents the zones 210 from adhering a second time, to the use of adhesives that require the zones 210 to be physically separated from the main unit 110 prior to being capable of removal from the tissues of the user. Any means suitable to prevent or discourage multiple uses of the main unit 110 is contemplated by the present invention.

A number of embodiments of the invention have been described. Nevertheless, it will be understood that various modifications may be made without departing from the spirit and scope of the invention. Accordingly, other embodiments are within the scope of the following claims.

What is claimed is:

1. A wound treatment device for providing and maintaining a microenvironment associated with a wound, said wound treatment device comprising:
a main unit formed from a first layer of material and a second layer of material sealed together along external edges;
said main unit defining an enclosed space;
at least one inlet in communication with the enclosed space;
a plurality of delivery openings in communication with the enclosed space and also in communication with the microenvironment associated with the wound; and
a plurality of exhaust outlets providing a unidirectional conduit between the microenvironment associated with the wound and an external environment; and
said microenvironment capable of receiving one or more gases via the enclosed space for the treatment of the wound.

2. The wound treatment device of claim 1, wherein the first layer of material and the second layer of material are mated at selective points within the edges to form pillows within the enclosed space.

3. The wound treatment device of claim 1, wherein the enclosed space is in communication with at least one inlet, said inlet sized and shaped to accept and secure at one end a hose.

4. The wound treatment device of claim 3, wherein the at least one inlet connects at a first end to a source for gas and at a second end to the main unit.

5. The wound treatment device of claim 4, wherein the source of gas is a mixture of two or more gases.

6. The wound treatment device of claim 1, wherein the plurality of delivery openings is solely within the second layer of material.

7. The wound treatment device of claim 1, wherein the plurality of delivery openings is sized and shaped to allow gas to pass from the enclosed space to the microenvironment associated with the wound.

8. The wound treatment device of claim 1, wherein the plurality of delivery openings is dispersed uniformly substantially throughout the second layer of the main unit, so that gas is delivered in a relatively uniform manner to the microenvironment associated with the wound.

9. The wound treatment device of claim 2, wherein the plurality of exhaust outlets lie within mated intersections of the first layer of material and the second layer of material forming a conduit between the microenvironment associated with the wound and the external environment.

10. The wound treatment device of claim 1, wherein the main unit includes at least one zone capable of receiving an adhesive or other mating material to removably affix the main unit to an area associated with the wound.

11. The wound treatment device of claim 10, wherein the at least one zone includes means for compromising integrity of the second layer of material upon removal of the main unit from the area associated with the wound.

12. The wound treatment device of claim 11, wherein the wound treatment device cannot be reused upon removal of the main unit from the area associated with the wound.

13. The wound treatment device of claim 1, wherein the main unit is comprised of one or more flexible materials that are hypoallergenic, disposable, biodegradable and exhibit low gas permeability.

14. A wound treatment device for providing and maintaining a microenvironment associated with a wound, said wound treatment device comprising:
a main unit formed from a first layer of material and a second layer of material sealed together along external edges and mated at selective points within the edges;
said main unit defining an enclosed space;
at least one inlet in communication with the enclosed space;
a plurality of delivery openings in communication with the enclosed space and also in communication with the microenvironment associated with the wound, said plurality of delivery openings;
a plurality of exhaust outlets providing a unidirectional conduit between the microenvironment associated with the wound and an external environment; and
said microenvironment capable of receiving one or more gases via the enclosed space for the treatment of the wound,
said plurality of delivery openings dispersed uniformly substantially throughout the second layer of material, so that the one or more gases are delivered in a relatively uniform manner to the microenvironment associated with the wound; and
said first layer of material including a non-adhering dressing material.

15. The wound treatment device of claim 14, wherein the main unit includes at least one zone along its perimeter capable of receiving means for mating to removably affix the main unit to an area associated with the wound.

16. The wound treatment device of claim 15, wherein the at least one zone includes means for compromising integrity of the second layer of material upon removal of the main unit from the area associated with the wound.

17. The wound treatment device of claim 11, wherein the means for compromising prevents reuse of the main unit upon removal of treatment device from the area associated with the wound.

18. The wound treatment device of claim 14, wherein the first layer of material and the second layer of material are comprised of hypoallergenic, flexible, disposable and biodegradable material having low gas permeability.

19. The wound treatment device of claim 14, wherein the first layer of material and the second layer of material are comprised of the same material.

20. The wound treatment device of claim 14, wherein the first layer of material and the second layer of material are comprised of different material.
